(19) United States
(12) Patent Application Publication
(10) Pub. No.: US 2011/0003816 A1
(43) Pub. Date: Jan. 6, 2011

(54) OPHTHALMIC COMPOSITION

(75) Inventors: Arindam Halder, Baroda (IN); Ajay Jaysingh Khopade, Baroda (IN); Subhas Balaram Bowmick, Baroda (IN)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: SUN PHARMA ADVANCED RESEARCH COMPANY LIMITED, Mumbai (IN)

(21) Appl. No.: 12/921,330

(22) PCT Filed: Mar. 9, 2009
(86) PCT No.: PCT/IN09/00162

§ 371 Date: Sep. 7, 2010

(30) Foreign Application Priority Data
Mar. 7, 2008 (IN) 477/MUM/2008

(51) Int. Cl. A61K 31/5377; A61K 47/38; A61P 27/02

(52) U.S. Cl. 514/236.2; 514/781

(57) ABSTRACT

An ophthalmic composition comprising therapeutically effective amount of a beta-blocker and a polymeric vehicle consisting essentially of a water soluble cellulose derivative and polyvinylpyrrolidone; wherein the composition is a clear aqueous solution with a viscosity id 20 cps to 60 cps.

The present invention relates to a long-acting ophthalmic composition comprising therapeutically effective amount of a beta blocker.

### BACKGROUND OF THE INVENTION

Glaucoma is an ocular disease characterized by an elevated intra-ocular pressure, which, if untreated, may lead to optic nerve head damage, causing irreversible loss of visual field, and eventually blindness. Since elevated intraocular pressure is the major risk factor of glaucoma, lowering it by various drugs is the mainstay of glaucoma therapy.

Currently five classes of drugs are available for use in patients with glaucoma, among which beta blockers are used predominantly. They lower the pressure in the eye by reducing the production of aqueous-humor.

Timolol, a non-selective beta-blocker, first approved by FDA for ocular use in 1978, is available as topically administrable compositions for glaucoma therapy—the major composition being aqueous ophthalmic solutions. But the disadvantage associated with aqueous liquid formulations is that a large percentage of the drug administered to the eye is lost due to lachrymal drainage. As a result; only a small portion of the dose administered remains in contact with the cornea for a few minutes and an even smaller fraction penetrates to the eye.

To overcome this disadvantage, a variety of gelling drug delivery systems comprising gel-forming polymers were developed, which systems undergo liquid-gel phase transition utilizing various mechanisms like increase in the ionic strength (U.S. Pat. Nos. 5,403,841 and 4,861,760), interaction with the enzyme-lysozyme present in tear fluids (U.S. Pat. No. 6,174,524), change in pH (U.S. Pat. Nos. 4,136,173 and 4,136,177) or change in temperature (U.S. Pat. Nos. 4,474,751; 4,474,752; and 4,188,373). They are topically administered as a liquid drop, which gels upon contact with the physiological liquid of the eye, the transition occurring at the contact site. The active ingredients supplied in the form of gel forming solutions have their own advantages and disadvantages. There are a number of manufacturing, storage, dispensing and usage constraints associated with gel-forming eye drops. Moreover, the unpleasant feel in the eye is also a disadvantage.

Further, some new compositions viz. gel formulations were developed which presents an improvement over gel-forming compositions. U.S. Pat. No. 5,397,657 discloses one such ophthalmic composition that combines two polymers to obtain desired residual viscosity. The residual viscosity which develops upon instillation in the eye is the factor which ultimately determines the compatibility and the residence time of the gel. The formulations possess good film forming properties due to polyvinylalcohol component, and bioadhesion due to carbomer component. Though this gel formulation presents an improvement over the previously described gel-forming compositions, the residual viscosity attained is high enough to show adhesive effects, thereby leading to compatibility problem such as patients' complaint of the feeling of a foreign body presence.

The U.S. Pat. No. 6,645,963 discloses an eye drop that does not use a gel forming component, yet provides sustained release of the active medicament. The composition uses short chain fatty acids, such as sorbic acid, to increase penetration and improve retention time in the eye tissues. The ocular tissues thus act as a drug storage site that prolongs drug action. The composition utilizes high concentration of sorbic acid to achieve this effect. The long term effects of high tissue concentration may be adverse.

U.S. Pat. No. 7,147,844, describes a system for stabilizing a lachrymal fluid layer over contact lenses, to remove dryness and unpleasantness in the eyes of contact lens wearers and provides a good, moist and instilling feel. The inventor found that polyvinylpyrrolidone is adsorbed on the ionic-contact lens, which in turn enhances its water retention capacity, and further use of a viscosity increasing agent like hydroxypropyl methylcellulose sustains the above mentioned improvement effects in the eye of the wearers. The composition possessed a kinematic viscosity of 1-8 mm²/sec. The invention provides composition which ensures comfort to the eyes of contact lens bearers without involving therapeutic applicability. This patent does not disclose any therapeutic ophthalmic compositions for once a day use.

U.S. Pat. No. 7,306,802 ('802 patent), U.S. Pat. No. 7,244,440 ('440 patent) and U.S. Pat. No. 7,329,411 ('411 patent) relates to ophthalmic compositions containing a synergistic combination of three polymers. The '802 patent provides an aqueous composition suitable for topical ophthalmic administration comprising three polymeric ingredients having a synergistic effect on the viscosity of the composition, wherein the three polymeric ingredients include hydroxypropylmethylcellulose and a combination of two polymers selected from the group of combinations consisting of guar-gum and a carboxyvinyl polymer; guar gum and hydroxyethyl cellulose, guar gum and dextran, hydroxyethyl cellulose and a carbovinyl polymer and dextran and a carbovinyl polymer. The composition is suitable for use as artificial tears, or as a vehicle for ophthalmic drugs. The composition of the present invention is not disclosed in said patents.

The '440 patent and '411 patent provides a method of alleviating the symptoms of dry eye comprising topical administration to the eye an aqueous composition as described by the '802 patent.

US Patent Application no.—20070128156 and 20040253280 describe an aqueous ophthalmic composition suitable for use as artificial tears or as vehicles for ophthalmic drugs, comprising a viscosity enhancing amount of a combination of two polymers having a synergistic effect on the viscosity of the composition, and wherein the combination of two polymers is selected from the group consisting of—hydroxypropyl methylcellulose and guar gum; hydroxypropyl methylcellulose and a carboxyvinyl polymer; hydroxypropyl methylcellulose and hydroxyethylcellulose; hydroxypropyl methylcellulose and hyaluronic acid; hyaluronic acid and a carboxyvinyl polymer; hyaluronic acid and guar gum; or a carboxyvinyl polymer and guar gum.

A formulation that eliminates above mentioned drawbacks of the eye drop formulations and combines the advantages of compatibility, improved penetration and ease of administration, with the beneficial properties of prolonged residence time, film-forming property and sustained release of the medicament in the eye, is the most preferred ophthalmic formulation for ocular therapy. The present invention provides such an ophthalmic composition.

It is a surprising and interesting finding of the invention that when cellulose derivatives for example, hydroxypropyhnethylcellulose and vinyl polymers for example, polyvinylpyrrolidone are combined in certain ratios, there occurs a synergistic increase in viscosity of the formulation, leading to good film-forming property and prolonged residence time, along with sustained release of the medicament and that such formulations are clear.

Additional aspects and advantages of the present invention will become apparent to those skilled in the art upon reading the detailed description, wherein only the preferred embodiment of the invention is revealed and explained, in a simple manner along with illustration of the best mode contemplated for carrying out the invention. As will be realized, the invention is capable of other and different embodiments, and its several details that are apparent, are capable of modifications in various respects, all without departing from the invention. Accordingly, the drawings and description are to be regarded as illustrative in nature, and not as restrictive.

### OBJECTS OF THE INVENTION

The present invention relates to a long-acting ophthalmic composition comprising therapeutically effective amount of a beta blocker.

It is an object of the present invention to provide a long-acting, sustained-release ophthalmic composition comprising a beta blocker which is suitable for once-a-day instillation.

It is another object of the invention to provide a once-a-day ophthalmic composition for reducing and controlling elevated intraocular pressure (IOP), especially the elevated IOP associated with glaucoma.

It is another object of the present invention to provide an ophthalmic composition which is a clear aqueous solution; is isotonic and compatible with the ocular fluids; has prolonged residence time; show good film-forming property; is non-irritating; possesses good antimicrobial properties.

### SUMMARY OF THE INVENTION

The present invention may be summarized as follows:

(A) The present invention provides an ophthalmic composition comprising therapeutically effective amount of a beta blocker and a pharmaceutically acceptable polymeric vehicle consisting essentially of a water soluble cellulose derivative and polyvinyl pyrrolidone, wherein the composition is a clear aqueous solution having a viscosity of 20 cps to 60 cps.

(B) An ophthalmic composition as defined in (A), wherein the beta-blocker is timolol maleate.

(C) An ophthalmic composition as defined in (A) comprising a therapeutically effective amount of timolol or its pharmaceutically acceptable salt, cellulose derivative whose 2% w/v aqueous solution has a viscosity in the range of about 3500 cps to about 5600 cps at 20° C. and polyvinyl pyrrolidone whose 10% w/v aqueous solution has a viscosity in the range of about 300 cps to about 700 cps at 20° C., wherein the composition is a clear aqueous solution having a viscosity of 20 cps to 60 cps and surface tension between 25 dynes/cm to 50 dynes/cm.

(D) An ophthalmic composition as defined in (A) or (C) wherein the cellulose derivative is selected from the group consisting of hydroxyethyl cellulose, hydroxypropyl cellulose, hydroxypropyl methylcellulose, methyl cellulose and mixtures thereof.

(E) An ophthalmic composition as defined in (A) or (C) wherein the cellulose derivative is hydroxypropyl methylcellulose and is used in combination with polyvinylpyrrolidone in a weight by weight ratio ranging from about 60:40 to about 20:80.

(E) An ophthalmic composition as defined in (A), wherein the polymer is present in a concentration of about 1.4% to about 5.0% by weight of the composition.

(F) An ophthalmic composition as defined in (A), wherein hydroxypropyl methylcellulose is used in an amount ranging from about 0.3% to about 2.0% by weight of the composition and polyvinylpyrrolidone is used in an amount ranging from about 1.0% to about 5.0% by weight of the composition.

(G) An ophthalmic composition as defined in (A) or (C), wherein the composition is suitable for once-a-day instillation.

(H) An ophthalmic composition as defined in (C), wherein the concentration of hydroxypropyl methylcellulose is 0.5% by weight and concentration of polyvinylpyrrolidone is 2.0% by weight of the composition.

(I) An ophthalmic composition as defined in (A), wherein the percent transmission is greater than 95% and the viscosity is greater than 35 cps.

(J) An ophthalmic composition comprising therapeutically effective amount of a beta blocker and a polymeric vehicle consisting essentially of a water soluble cellulose derivative and polyvinyl pyrrolidone, wherein the ratio of the water soluble cellulose derivative and polyvinyl pyrrolidone ranges from 60:40 to 20:80 and the concentration of the said polymer ranges from 1.4% to 5.0% by weight of the composition.

(K) An ophthalmic composition as defined in J wherein the water soluble polymer is selected from the group consisting of hydroxypropyl methyl cellulose, hydroxyethyl cellulose and hydroxypropyl cellulose.

(L) An ophthalmic composition as defined in K wherein the molecular weight of the polyvinyl pyrrolidone is 1,000,000 to 1,500,000.

(M) An ophthalmic composition as defined in L wherein the polymeric vehicle consist essentially of hydroxypropyl methyl cellulose of 3500 cps to 5600 cps viscosity of 2% by weight of aqueous solution and polyvinyl pyrrolidone of 300 cps to 700 cps viscosity of 10% w/v aqueous solution.

### BRIEF DESCRIPTION OF FIGURE

FIG. 1: It represents a comparative account of concentration-time profile of drug retained in aqueous humor, on topical application of the ophthalmic composition prepared according to Example 3 with the marketed product NYOGEL composition (Novartis Pharmaceuticals Ltd.,) having timolol maleate in a concentration of 0.1% by weight of the composition.

### DETAILED DESCRIPTION OF THE INVENTION

The present invention provides an ophthalmic composition comprising therapeutically effective amount of a beta blocker and a pharmaceutically acceptable polymeric vehicle consisting essentially of a water soluble cellulose derivative and polyvinyl pyrrolidone, wherein the composition is a clear aqueous solution having a viscosity of 20 cps to 60 cps.

The ophthalmic compositions of the present invention are characterized as being clear aqueous solutions. These “clear aqueous solutions” as stated herein, are defined as those solutions which do not cause any visual disturbance and/or do not affect vision, upon topical instillation to the eye and when examined under suitable conditions of visibility, are practically clear and practically free from particles. Ophthalmic compositions containing polymers which show percent transmission greater than 90% are referred to as ‘clear aqueous solutions’.

The term ‘percent transmission’ as used herein is defined below: When light is allowed to pass through the ophthalmic composition of the present invention, the percentage of incident light which is transmitted through the solution is referred to as “Percent Transmission”. As mentioned, the property of “Percent Transmission” relates to the clarity of the aqueous solution or composition. The clarity of the composition is poor if percent transmission is less than 85%. Preferably the percent transmission is greater than 90%. Generally, the percent transmission is determined at a wavelength of about 650 nm, but any other suitable wavelength may be selected for determining the clarity of the solution.

The compositions of the present invention comprising pharmaceutically acceptable polymeric vehicle are further characterized by possessing a viscosity of 20 cps to 60 cps (centipoises per second); preferably the pharmaceutically acceptable polymer is used in an amount to provide synergistic viscosity.

The term ‘Synergistic viscosity’ as used herein refers to the viscosity attained by the composition of the present invention (consisting of a combination of a water soluble cellulose derivative (A) and polyvinyl pyrrolidone (B) such that the viscosity attained (in cps) is more than the sum of viscosities of two aqueous compositions ‘A’ and ‘B’, wherein ‘A’ contains only cellulose derivative and ‘B’ contains only vinyl derivative.

To achieve a target viscosity for a topically administrable ophthalmic composition, one approach could be to simply add a sufficient amount of one polymeric ingredient. This however may require use of large amount of that polymer, which can be undesirable. Instead, it is beneficiary and desirable to minimize the total amount of polymeric ingredients in topical ophthalmic compositions. So another approach comprising use of a mixed polymer system, containing two or more polymers that can interact in such a way so as to provide synergism in viscosity, can lead to attainment of the target viscosity at a comparatively very lower amount of the total polymer required, thus also reducing the cost of material.

The present invention provides an ophthalmic compositions comprising a pharmaceutically acceptable a polymeric vehicle that consist essentially of a mixture of two category of polymers i.e water soluble cellulose derivatives and polyvinyl pyrrolidone in such grades, ratios and concentrations that provide a synergistic viscosity of 20 cps to 60 cps and also imparts greater than 95% clarity to the solution which is desirable as the composition is topically administered. The synergistic viscosity of 20 cps to 60 cps, attained by the composition of the present invention, when the two polymers used, for example, hydroxypropylmethylcellulose and polyvinylpyrrolidone, are combined in certain ratios, is useful in prolonging the retention (residence) time of the formulation at the surface of the eye, and sustaining release of the medicament from it, leading to prolonged action of the medicament, and thereby facilitating once-a-day administration.

The present invention provides an ophthalmic composition that does not show a significant change in residual viscosity upon installation which may minimize inter-patient variation. Residual viscosity develops on eye as a result of mixing of eye drop with tear fluid.

Although the ophthalmic composition of the present invention exhibits a surface tension almost same as artificial tear fluid, the viscosity exhibited by the compositions is about 20 cps to 60 cps in contrast to the artificial tear fluids that are known in the art.

Without wishing to be bound by theory, the applicant believes that Polyvinyl pyrrolidone used in the composition of the present invention, may be interacting with water soluble cellulose derivative though hydrogen bonding interactions forming an interpenetrated network. It is the formation of interpenetrated network that may lead to synergistic viscosity increase thereby providing a matrix for sustained release of drugs. The interactions may be so significant that depending on the polymer ratio and concentration, liquid coacervate formation/phase separation may occur leading to reduced clarity of the composition. Surprisingly, the composition of the present invention demonstrates a significant synergistic viscosity yet is clear solution not showing a phase separation. An ophthalmic composition comprising therapeutically effective amount of a beta blocker and a polymeric vehicle consisting essentially of a water soluble cellulose derivative and polyvinyl pyrrolidone, wherein the ratio of the water soluble cellulose derivative and polyvinyl pyrrolidone ranges from 60:40 to 20:80 and the concentration of the said polymer ranges from 1.4% to 5.0% by weight of the composition.

The ophthalmic composition of the present invention comprises therapeutically effective amount of a therapeutic agent useful for the treatment of glaucoma (anti-glaucoma agents).These include but, are not limited to, beta-blockers such as timolol, betaxolol, levobetaxolol, carteolol, their derivatives, salts and mixtures thereof. In one embodiment of the present invention, the therapeutic agent used is a salt of timolol. In preferred embodiment of the present invention, the therapeutic agent used is timolol maleate.

Timolol, a non-selective beta-adrenergic blocker, having a molecular weight of 432.50, when applied topically as an ophthalmic solution, reduces the intraocular pressure in the eye. It is thus indicated in patients with ocular hypertension or open angle glaucoma. It also shows certain systemic effects which includes (1) Beta-adrenergic receptor blockade in the heart causing reduction in cardiac output in both healthy subjects and patients with heart disease and (2) Beta-adrenergic receptor blockade in the bronchi and bronchioles resulting in increased airway resistance from unopposed parasympathetic activity. Therefore, the drug must be used with caution in patients in whom beta-adrenergic blockade may be undesirable. Timolol for glaucoma therapy is thus contraindicated in patients with compromised pulmonary functions and in patients who cannot tolerate its systemic cardiovascular action. Timolol maleate is used in the compositions of the present invention in therapeutically effective amounts. Timolol maleate may be used in an amount ranging from about 0.01% to about 2.0% by weight of the composition, preferably from about 0.05% to about 1.0% by weight of the composition and most preferably from about 0.1% to about 0.5% by weight of the composition.

The ophthalmic composition comprises pharmaceutically acceptable polymer vehicle consisting essentially of a mixture of water soluble cellulose derivatives such as hydroxypropylmethylcellulose, hydroxyl ethylcellose, hydroxypropyl cellulose, methyl cellulose and polyvinylpyrrolidone.

In one embodiment of the present invention, the pharmaceutically acceptable polymeric vehicle is a mixture of hydroxypropylmethylcellulose used as the water soluble cellulose derivative and polyvinylpyrrolidone.

The hydroxypropyl methylcellulose (IIPMC) used in the compositions of the present invention is a cellulose polymer, in particular, propylene glycol ether of methylcellulose. It functions to provide the desired level of viscosity via synergism with PVP and also shows demulcent activity. HPMC is available in a variety of grades under several trade names. The various grades differ in methoxy and hydroxypropyl content, as well as in terms of molecular weight and viscosity (of 2% solution in water at 20° C.). The cellulose ether that can be used in the compositions of the present invention may be selected from any available grade of HPMC. Suitable material is sold by the ‘The Dow Chemical Company’ (“Dow”) under the trademark METHOCEL. The HPMC grade which may be selected to be used in the compositions of the present invention include, but is not limited to:

METHOCEL E, (USP grade 2910/HYPROMELLOSE 2910) including (a) METHOCEL E3 (Premium LV) having 28-30 weight percent methoxyl content, 7-12 weight percent hydroxypropyl content and viscosity of a 2% aqueous solution of 2.4-3.6 cps (b) METHOCEL E5 (Premium LV) having 28-30 weight percent methoxyl content, 7-12 weight percent hydroxypropyl content and viscosity of a 2% aqueous solution of 4.0-6.0 cps. (c) METHOCEL E6 (Premium LV) having 28-30 weight percent methoxyl content, 7-12 weight percent hydroxypropyl content and viscosity of a 2% aqueous solution of 5.0-7.0 cps (d) METHOCEL E15 (Premium LV) having 28-30 weight percent methoxyl content, 7-12 weight percent hydroxypropyl content and viscosity of a 2% aqueous solution of 12.0-18.0 cps (e) METHOCEL E50 (Premium LV) having 28-30 weight percent methoxyl content, 7-12 weight percent hydroxypropyl content and viscosity of a 2% aqueous solution of 40.0-60.0 cps (f) METHOCEL E4M (Premium) having 28-30 weight percent methoxyl content, 7-12 weight percent hydroxypropyl content and viscosity of a 2% aqueous solution of 3000.0-5600.0 cps (g) METHOCEL E10M (Premium CR) having 28-30 weight percent methoxyl content, 7-12 weight percent hydroxypropyl content and viscosity of a 2% aqueous solution of 7500.0-14000.0 cps.

METHOCEL F, (USP grade 2906/HYPROMELLOSE 2906) including (a) METHOCEL. F50 (Premium) having 27-30 weight percent methoxyl content and 4-7.5 weight percent hydroxypropyl content. (b) METHOCEL F4M (Premium LV). METHOCEL K, (USP grade 2208/HYPROMELLOSE 2208) including (a) METHOCEL K3 (Premium LV) having 19-24 weight percent methoxyl content, 4-12 weight percent hydroxypropyl content and viscosity of a 2% aqueous solution of 2.4-3.6 cps (b) METHOCEL K100 (Premium LV) having 19-24 weight percent methoxyl content, 4-12 weight percent hydroxypropyl content and viscosity of a 2% aqueous solution of 80.0-120.0 cps (c) METHOCEL K4M (Premium) having 19-24 weight percent methoxyl content, 4-12 weight percent hydroxypropyl content and viscosity of a 2% aqueous solution of 3000.0-5600.0 cps (d) METHOCEL K15M (Premium) having 19-24 weight percent methoxyl content, 4-12 weight percent hydroxypropyl content and viscosity of a 2% aqueous-solution of 11,250.0-21,000.0 cps (e) METHOCEL K100M (Premium) having 19-24 weight percent methoxyl content, 4-12 weight percent hydroxypropyl content and viscosity of a 2% aqueous solution of 80,000.0-120,000.0 cps.

METHOCEL A15 (Premium LV); METHOCEL A4C (Premium); METHOCEL A15C (Premium); METHOCEL A4M (Premium), HPMC USP Grade 1828 having 16.5-20 weight percent methoxyl content, 23-32 weight percent hydroxypropyl content.

Most preferred grade for use in the compositions of the present invention is METHOCEL E4M (USP 2910), characterized by having: 28-30 weight percent methoxyl content, 7-12 weight percent hydroxypropyl content and viscosity of a 2% aqueous solution of 3500-5600 cps (centipoises per second).

It is to be understood, however, that the invention is not limited to any specific hydroxypropyl methylcellulose; and that any equivalent hydroxypropyl methylcellulose of pharmaceutical grade may be used to achieve equivalent results.

In embodiments where hydroxypropyl methyl cellulose is used, it is present in an amount ranging from about 0.05% to about 8.0% by weight of the composition, preferably in an amount ranging from about 0.1% to about 4.0% by weight of the composition, more preferably in an amount ranging from about 0.3% to about 2.0% by weight of the composition, the amount being varying depending upon the grades of the polymer used.

In embodiments where water soluble polymer is hydroxypropyl methyl cellulose and the polyvinyl pyrrolidone, the two polymers may be used in a weight by weight ratio ranging from about 95:5 to about 5:95, preferably 60:40 to about 20:80. The two polymers may be present in the compositions in an amount ranging from about 0.1% to about 10.0% by weight of the composition, preferably in an amount ranging from about 1.0% to about 8.0% by weight of the composition, more preferably in an amount ranging from about 1.0% to about 5.0% by weight of the composition; the amount being varying depending upon the grades of the polymer used.

According to another embodiment of the present invention, the water soluble cellulose derivative used is hydroxyethyl cellulose. Hydroxyethyl cellulose is a nonionic, water soluble polymer. It is commonly known as Cellosize and Natrosol. The polymer is primarily used as a thickening agent in ophthalmic and topical formulations. It is available in wide range of viscosity types, eg. Cellosize is manufactured in eleven regular viscosity grades. Hydroxyethyl cellulose grades differ principally in their aqueous solution viscosities which range from 2-200 mPas for a 2% w/v aqueous solution. Two types if Cellosize are produced, a WP-type which is a normal dissolving material and a QP-type, which is a rapid-dispersing material. The lowest viscosity grade is available only in the WP-type. Five viscosity grades (09, 3, 40,300 and 4400) are produced in both WP-and QP-types. Natrosol 250 has a degree of substitution of 2.5 and is produced in ten viscosity types. The hydroxyethyl cellulose in the polymeric vehicle is present in amount ranging from 0.1% to 2% w/v, preferably 0.2% to 0.6% and most preferably about 0.5% w/v. In one preferred embodiment, the hydroxyethyl cellulose and polyvinyl pyrrolidone are used in a ratio of 1:4. When the ratio of hydroxyethyl cellulose to polyvinyl pyrrolidone is about 1:4, the viscosity of polyvinyl pyrrolidone used is preferably more than 3 cps, preferably, preferably more than 300 cps and most preferably about 700 cps. It was found that when the ratio of these two polymers is about 1:4, a synergistic effect on the viscosity is prominent when polyvinyl pyrrolidone of high viscosity i.e 90 K is used.

In yet another embodiment of the present invention, the polymeric vehicle comprises hydroxypropyl cellulose as the water soluble cellulose derivative. In another embodiment, methylcellulose is used as a water soluble cellulose derivative.

The polymeric vehicle of the composition of the present invention comprises another pharmaceutically acceptable polymer, polyvinylpyrrolidone (PVP), a tertiary amide polymer. This is a linear polymer of 1-vinyl-2-pyrrolidone groups, in which the degree of polymerization results in polymers of various molecular weights. It is characterized by its viscosity in aqueous solution, relative to that of water, expressed as a K-value, which ranges from 10 to 120, constituting its various grades. The polyvinylpyrrolidone that can be used in the compositions of the present invention may be selected from any of the available grade of polyvinylpyrrolidone. Such materials are sold by ISP Technologies, Inc., under the trademark PLASDONE.

The PVP grade which may be selected to be used in the compositions of the present invention include, but is not limited to:

PVP K-11/14, whose 10% w/v aqueous solution has a dynamic viscosity in the range of about 1.3 cps to about 2.3 cps at 20° C.,

PVP K-16/18, whose 10% w/v aqueous solution has a dynamic viscosity in the range of about 1.5 cps to about 3.5 cps at 20° C.,

PVP K-24/27, whose 10% w/v aqueous solution has a dynamic viscosity in the range of about 3.5 cps to about 5.5 cps at 20° C.,

PVP K-28/32, whose 10% w/v aqueous solution has a dynamic viscosity in the range of about 5.5 cps to about 8.5 cps at 20° C.,

PVP K-85/95, whose 10% w/v aqueous solution has a dynamic viscosity in the range of about 300.0 cps to about 700.0 cps at 20° C.,

The preferred grades which can be used in the compositions of the present invention include PVP K-30, PVP K-60 and PVP K-90. The most preferred grade used in the compositions of the present invention is PVP K-90, whose 10% w/v aqueous solution has a dynamic viscosity in the range of about 300.0 cps to about 700.0 cps at 20° C., and has an approximate molecular weight of about 1,000,000 to 1,500,000.

It is to be understood, however, that the invention is not limited to any specific polyvinylpyrrolidone; and that any equivalent polyvinylpyrrolidone of pharmaceutical grade, may be used to achieve equivalent results.

Polyvinylpyrrolidone (PVP) is chosen because it is particularly useful as a wetting agent, besides acting as a viscosifying agent PVP has a number of other characteristics that makes it useful in combination with the various well known components in ophthalmic solutions. Polyvinylpyrrolidone acts as a detoxicant, binding anti-toxins present in eye fluids and rendering them harmless. Additionally, PVP acts as a demulcent lubricant by means of a combination of adhesive and lubricating properties that aid in the spreading of the viscous solution over the eye. PVP provides tear film stability and wetting of the corneal surfaces, and also allows the use of benzallconium chloride in effective preservative concentrations in the solution. PVP may be used in the compositions of the present invention in an amount ranging from about 0.01% to about 10.0% by weight of the composition, preferably in an amount ranging from about 0.1% to about 8.0% by weight of the composition, more preferably in an amount ranging from about 1.0% to about 5.0% by weight of the composition, the amount being varying depending upon the grades of the polymer used.

In one embodiment of the present invention, a preferred combination of hydroxypropylmethylcellulose (HPMC) and polyvinylpyrrolidone (PVP) that can be used in the compositions of the present invention is HPMC whose 2% w/v aqueous solution has a viscosity in the range of about 3500 cps to about 5600 cps at 20° C. (called HPMC E4M) and polyvinylpyrrolidone whose 10% w/v aqueous solution has a viscosity in the range of about 300 cps to about 700 cps at 20° C. (called PVP K90). This combination may be used preferably in a weight by weight ratio (HPMC E4M: PVP K90) ranging from about 80:20 to about 10:90.

In preferred embodiment of the present invention, the composition comprises HPMC E4M in an amount 0.5% by weight of the composition and polyvinylpyrrolidone K-90 in an amount 2.0% by weight of the composition. This combination may be used in a weight by weight ratio (HPMC E4M:PVP K90) ranging from about 80:20 to about 10:90, wherein the viscosity is about 25 cps, preferably about 40 cps and the percent transmission is greater than 90%, preferably greater than 95%.

The present invention provides a long-acting, ophthalmic composition suitable for once-a-day instillation. The compositions prepared according to the present invention were tested for the efficacy in reducing the intraocular pressure in glacoumatous rabbit model. The compositions prepared according to the invention were found reduce the increased intraocular pressure reduction from up to 15-18 h and was comparable to the marketed once a day compositions. The compositions of the present invention were also found to be safe in terms of irritation to the cornea, iris and conjunctivae.

The compositions of the present invention may further comprise pharmaceutically acceptable excipients conventional to the pharmaceutical art. Typical of such pharmaceutically acceptable excipients include osmotic/tonicity-adjusting agents, preservatives, one or more pharmaceutically acceptable buffering agents and pH-adjusting agents, solubilizing agents, vehicles and other agents conventional in art that may be used in formulating an ophthalmic composition.

The ophthalmic compositions are required to be isotonic with respect to the ophthalmic fluids present in the human eye. These solutions are characterized by osmolalities of 250-375 mOsm/kg. Osmolality of the solutions is adjusted by addition of an osmotic/tonicity adjusting agent. Osmotic agents that may be used in the compositions of the present invention to make it isotonic with respect to the ophthalmic fluids present in the human eye, are selected from the group comprising sodium chloride, potassium chloride, calcium chloride, sodium bromide, mannitol, glycerol, sorbitol, propylene glycol, dextrose, sucrose, and the like, and mixtures thereof. In preferred embodiments of the present invention, mannitol is used as the osmotic agent. Mannitol may be present in the compositions of the present invention in an amount ranging from about 2.0% to about 6.0% by weight of the composition, preferably from about 3.0% to about 5.0% by weight of the composition and most preferably in an amount of about 4.5% by weight of the composition.

Further, the ophthalmic compositions of the present invention may comprise preservatives in effective amounts. Antimicrobial effective amounts of a preservative may be determined by performing preservative efficacy tests or antimicrobial effectiveness tests. These tests are inter alia described in chapter 51 of the United States Pharmacopoeia 29-National Formulary 24 (USP 29-NF 24). The preservatives may be used in an amount within the concentration ranges described in standard reference books like ‘Remington's Pharmaceutical Sciences’ and ‘Handbook of Pharmaceutical Excipients’.

The preservative may be selected from: Quaternary ammonium compounds such as benzalkonium chloride (BKC) and benzethonium chloride; Organic mercurials such as phenylmercuric acetate, phenylmercuric nitrate and thimerosal; Parabens such as methyl and propyl paraben; ethyl paraoxybenzoate or butyl paraoxybenzoate; Acids and their pharmaceutically acceptable salts such as sorbic acid, potassium sorbate, boric acid, borax, salicylic acid; Substituted alcohols and phenols such as chlorobutanol, benzyl alcohol; phenyl ethanol; Amides such as acetamide; and the like, and combinations thereof. Preferably the ophthalmic compositions of the present invention comprise ‘quaternary ammonium compound’ as a preservative, particularly benzalkonium chloride. Benzalkonium chloride is characterized as a mixture of alkyldimethyl benzylammonium chlorides. It is employed in the compositions of the present in a preferable concentration of about 0.01 to about 0.02% by weight of the composition.

According to another embodiment of the present invention, the ophthalmic composition may be self preserving. The ingredients that make the composition self preserving include, but are not limited to, combination of zinc salts and boric acid in presence of tromethamine.

In order to achieve, and subsequently maintain, an optimum pH, the ophthalmic compositions essentially contain a pH adjusting agent and/or a buffering agent. The preferred range of pH for an ophthalmic formulation is about 6.8 to about 7.8, and the most preferred pH is about 7.4.

The ophthalmic compositions of the present invention comprise a pharmaceutically acceptable pH adjusting agent that may be selected from the group comprising acetic acid or salts thereof, boric acid or salts thereof, phosphoric acid or salts thereof; citric acid or salts thereof, tartaric acid or salts thereof, sodium hydroxide, potassium hydroxide, sodium carbonate, sodium hydrogencarbonate, trometamol, and the like and mixtures thereof. Particularly, preferred pH adjusting agents that may be used in the composition of the present invention include acetic acid, hydrochloric acid, sodium carbonate and sodium hydroxide. These agents are used in amounts necessary to produce a pH ranging from about 6.0 to about 8.0.

Besides above mentioned ingredients, the formulation of the invention may include a number of additional components to provide various effects, as is well known in this field. For example, the composition may include edetate disodium, which may function as a co-preservative and chelating agent.

The ophthalmic compositions of the present invention may be prepared by following a general method described below:

Take WFI in two sets of stainless steel (SS316) vessels each fitted with an overhead stirrer. Disperse the two polymers HPMC and PVP K-90 gradually in WFI in the two vessels separately, under stirring, to obtain homogeneous dispersions. Prepare a solution of the therapeutically active agent Timolol Maleate and the osmotic agent (mannitol, if present), in the WFI. Add this to the PVP K-90 dispersion under stirring. Mix homogeneously the HPMC phase with the PVP K-90 dispersion under stirring. With the stirring being continued, add the preservative (benzalkonium chloride) solution to this. Adjust the pH to about 7.4 with acetic acid or sodium hydroxide. Make up the volume to 100% with WFI. Autoclave the bulk solution for 20 min at 121° C., cool and filter if required, This results in the desired ophthalmic composition of the present invention.

In one embodiment of the present invention, the ophthalmic composition is preservative free. It is prepared by first making the polymeric vehicle for example, hydroxypropyl Methyl Cellulose K100M (HPMC K100M) and PVP K 30 dissolved separately in water for injection under stirring. The two polymer phases were mixed with stirring and autoclaved at 121° C. for 20 min followed by cooling to room temperature under stirring. Stirring should be continued until all of the cloudy, swollen or gelatinized particles are dissolved (Phase I, polymer phase). Timolol Maleate, Tromethamine, sodium chloride, boric acid and zinc chloride were dissolved sequentially in WFI at 20-25° C. with continuous stirring (Phase II, drug phase). The drug phase was mixed with the solution of phase I (polymer phase) under stirring aseptically through 2.0-0.2μ filters in series. Finally the volume was adjusted to 100% with WFI and filtered under aseptic conditions through 2.0 micron filter.

The exact procedure varies slightly depending upon the ingredients used. The detailed description is included in the examples.

The present invention provides a method of treatment of glaucoma, comprising once-a-day administration of the ophthalmic composition of the present invention comprising a therapeutic agent (beta-blocker), topically to the eye and attaining sustained release of the active agent, thereby reducing and controlling the elevated intraocular pressure, especially the elevated IOP associated with glaucoma.

While the present invention is disclosed generally above, additional aspects are further discussed and illustrated with reference to the examples below. However, the examples are presented merely to illustrate the invention and should not be considered as limitations thereto.

### Example 1

An ophthalmic composition for once-a-day instillation, according to the present invention is shown in Table 1.

TABLE 1

| S. No. | Ingredients | Concentration (% w/v) |
|---|---|---|
| 1. | Timolol Maleate | 0.5 |
| | (equivalent to Timolol) | |
| 2. | Hydroxypropylmethylcellulose E4M | 0.5 |
| | (HPMC-E4M) | |
| 3. | Polyvinylpyrrolidone K-90 (PVP K-90) | 2.0 |
| 4. | Sodium carbonate | 0.5 |
| 5. | Benzalkonium chloride solution | 0.02 |
| 6. | Acetic acid | q.s |
| 7. | Water for Injection (WFI) | q.s. |

WFI was taken in two sets of stainless steel (SS316) vessels each fitted with an overhead stirrer. HPMC-E4M and PVP K-90 was dispersed gradually in WFI in the two vessels separately, under stirring, to obtain homogeneous dispersions. In another beaker, Sodium Carbonate was dissolved in required volume of WFI to get a preconcentrate (10% w/v) solution. To this, the drug Timolol Maleate was added and it is kept for 1 h to form an oily base of Timolol base. The oily base so obtained was added to the PVP K-90 dispersion under stirring, the stirring being continued until the oily droplets' dissolve. The HPMC-E4M phase was mixed homogenously with the PVP K-90 dispersion under stirring. Further, benzalkonium chloride solution (BKC solution) was added with stirring and the pH was adjusted to about 7.4 with acetic acid. Finally the volume was adjusted to 100%. with WFI; the bulk solution was autoclaved for 20 min at 121° C. and cooled to obtain the final formulation.

### Example 2

An ophthalmic composition for once-a-day instillation, according to the present invention is shown in Table 2.

TABLE 2

| | | Concentration |
|---|---|---|
| S. No. | Ingredients | (% w/v) |
| 1. | Timolol Maleate | 0.5 |
| | (equivalent to Timolol) | |
| 2. | Hydroxypropylmethylcellulose E4M (HPMC- | 0.5 |
| | E4M) | |
| 3. | Polyvinylpyrrolidone K-90 (PVP K-90) | 2.0 |
| 4. | Benzalkonium chloride solution | 0.02 |
| 5. | Acetic acid | 0.05 |
| 6. | Sodium Hydroxide | q.s. |
| 7. | Water for Injection(WFI) | q.s. |

Water for injection was taken in two sets of stainless steel (SS316) vessels each fitted with an overhead stirrer. HPMC-E4M and PVP K-90 was dispersed gradually in WFI in the two vessels separately, under stirring, to obtain homogeneous dispersions. In another beaker, the drug Timolol Maleate was dissolved in required quantity of WFI. The drug solution was added to the PVP K-90 dispersion under stirring followed by addition of required amount of acetic acid with stirring. The pH was adjusted to 7.0 with sodium hydroxide solution. The PVP K-90 dispersion was mixed homogenously with the HPMC-E4M phase, followed by addition of BKC solution under stirring. The pH was adjusted to 7.4 with sodium hydroxide solution. Finally the volume was adjusted to 100% with WFI. The bulk solution was filtered through 2-20 micron glass fiber filter and then autoclaved for 20 min at 121° C. The solution was then cooled to room temperature obtain the final formulation.

### Example 3

An ophthalmic composition for once-a-day instillation, according to the present invention is shown in Table 3

TABLE 3

| | | Concentration |
|---|---|---|
| S. No. | Ingredients | (% w/v) |
| 1. | Timolol Maleate (equivalent to | 0.1 |
| | Timolol) | |
| 2. | Hydroxypropylmethylcellulose E4M | 0.5 |
| | (HPMC-E4M) | |
| 3. | Polyvinylpyrrolidone K-90 (PVP K- | 2.0 |
| | 90) | |
| 4. | Benzalkonium chloride solution | 0.02 |
| 5. | Acetic acid | 0.05 |
| 6. | Sodium Hydroxide | q.s. |
| 7. | Water for Injection(WFI) | q.s. |

The ophthalmic composition of this example 3 was prepared by following the same method as described in example 2 above.

### Example 4

An ophthalmic composition for once-a-day instillation, according to the present invention is shown in Table 4.

TABLE 4

| | | Concentration | |
|---|---|---|---|
| | | (% w/v) | |
| S. No. | Ingredients | Example 4a | Example 4b |
| 1. | Timolol Maleate | 0.5 | 0.5 |
| | (equivalent to Timolol) | | |
| 2. | Hydroxypropylmethylcellulose | 0.5 | 0.5 |
| | E4M (HPMC E4M) | | |
| 3. | Polyvinylpyrrolidone K-90 | 2.0 | 2.0 |
| | (PVP K-90) | | |
| 4. | Mannitol | 4.5 | 4.5 |
| 5. | Benzalkonium chloride solution | 0.02 | 0.02 |
| 6. | Acetic acid | 0.05 | — |
| 7. | Tromethamine | — | q.s. |
| 8. | Sodium Hydroxide | q.s. | — |
| 9. | Water for Injection (WFI) | q.s. | q.s. |

The ophthalmic composition of this example 4a was prepared by following the same method as described in example 2 above including the mannitol dissolution step in the drug phase.

The ophthalmic composition of this example 4b was prepared as follows: Water for injection was taken in two sets of stainless steel (SS316) vessels each fitted with an overhead stirrer. HPMC-E4M and PVP K-90 was dispersed gradually in WFI in the two vessels separately, under stirring, to obtain homogeneous dispersions. Mannitol was dissolved in PVP phase. The PVP phase was added to HPMC phase and the mixture was mixed thoroughly and filtered though 2-20 micron glass fiber filter and autoclaved at 121° C. for 20 min, followed by cooling to room temperature. In another beaker, the drug Timolol Maleate was dissolved in required quantity of WFI followed by addition of BKC solution with stirring. This solution was filtered aseptically through 0.2 micron nylon filter and added to the autoclaved polymer phase under stirring. The pH was adjusted to 7.4 with tromethamine solution. Finally the volume was adjusted to 100% with WFI.

The formulations prepared according to example 4a and 4b were subjected to accelerated stability conditions. The timolol content was determined to check the chemical stability of the compositions. The compositions were found to be stable at room temperature.

The formulations prepared according to example 4a and 4b has a viscosity of about 47.87 and 44.21 cps and % transmission of 98.43 and 100.0% respectively. A residual viscosity of the formulation of example 4b was performed. Ten ml of formulation was mixed with 0.45 ml of 20% sodium chloride solution. After mixing viscosity of the solution was measured on a Brookfield viscometer as described elsewhere in this patent. The viscosity was 42.90 cps which was 40.68 cps after mixing with saline. There was no significant change in viscosity. The surface tension of the formulations prepared according to example 4a and 4b determined on a Kruss K12MK6 tensiometer (Plate method) maintaining process temperature at 20-25 deg C. were approximately 30-32 dynes/cm, and approximately 35-40 dynes/cm , respectively.

Composition prepared according to the Example 4a was subjected to efficacy studies. The efficacy was determined by testing the reduction in the intraocular pressure in glaucomatous rabbits. Chronic ocular hypertension in rabbits was induced by a single injection of alpha-chymotrypsin into the posterior ocular chambers in animals. Achieving the steady elevated intraocular pressure, 70 μl l of test formulation was instilled into the left eye of each animal. After instillation of the composition, the intraocular pressure was measured at different time point i.e 30 min, 1, 2, 4, 6, 8 and 24 hours using a pneumatotonometer, Model 30 Classic (Reichert, USA). The % reduction in the intraocular pressure was calculated by comparing with initial readings. It was observed that the test composition of example 4a significantly reduced the intraocular pressure for about 15-18 hours and was comparable to the marketed compositions 1 and 2 in glaucomatous rabbits.

The test compositions were also subjected to safety studies. The two formulations were tested for eye irritation test in New Zealand Rabbits. The study involved single ocular instillation (100 μl with the help of micro pipette) into the right eye and the same volume of its placebo in the left eye of each of the three rabbits. Rabbits were examined immediately and after instillation of the compositions for 4, 24, 48 and 72 hours post instillation to note the signs/symptoms of eye irritation, if any. No sign of irritancy in cornea, iris and conjunctivae was noticed.

### Example 5

Studies were carried out by varying the ratio of HPMC-E4M and PVP K-90, (keeping the concentration of HPMC-E4M constant at 0.5%w/v), and their effects on the viscosity of the formulation, and on the percent transmission were determined. The percent transmission and viscosity of the formulation was determined by the methods given below.

Determination of Percent Transmission: Optical Study

The optical study involving the determination of percent transmission of the formulation was carried out using the UV-1700 Spectrophotometer, Shimadzu, Japan. The result obtained for various formulations is shown Table 5 and 6.

Determination of Viscosity: Rheological Study.

The rheological study involving the determination of viscosity of the formulation was carried out using the Brookfield dv-(iii) Ultra Programmable Rheometer (Spindle CPE 40, Entry Code: 40).

Procedure—0.5 g of formulation was taken in rheometer cup and a spindle was immersed in the test fluid through a calibrated spring which measures viscosity by sensing the torque required to rotate the spindle at constant speed while immersed in the sample fluid. The torque is proportional to the viscous drag on the immersed spindle, and thus to the viscosity of the fluid. The viscous drag of the fluid against the spindle was measured by the spring deflection. Spring deflection was measured with a rotary transducer. By rotating the spindle at several different speeds, shear dependent behavior of fluids can be also detected and analyzed.

The Table 5 below describes the various compositions obtained by varying the ratio of HPMC-E4M: PVP K-90 from 100:0 to 0:100. It also includes the determined values of percent transmission and viscosity (in cps) for the corresponding compositions.

TABLE 5

| | Concentration (% w/v) | | | | | | | | | |
|---|---|---|---|---|---|---|---|---|---|---|
| | HPMC | HPMC | HPMC | HPMC | HPMC | HPMC | HPMC | HPMC | | |
| | E4M:PVP | E4M:PVP | E4M:PVP | E4M:PVP | E4M:PVP | E4M:PVP | E4M:PVP | E4M:PVP | HPMC E4M:PVP | |
| | K90- | K90- | K90- | K90- | K90- | K90- | K90- | K90- | K90- | |
| Ingredients | 100:0 | 80:20 | 60:40 | 50:50 | 40:60 | 20:80 | 15:85 | 10:90 | 0:100 | |
| Hydroxypropyl | 0.5 | 0.5 | 0.5 | 0.5 | 0.5 | 0.5 | 0.5 | 0.5 | 0 | |
| methylcellulose | | | | | | | | | | |
| E4M | | | | | | | | | | |
| Polyvinyl | 0 | 0.13 | 0.3 | 0.5 | 0.8 | 2.0 | 2.83 | 4.5 | 0.5 | 2.0 |
| pyrrolidone | | | | | | | | | | |
| K90 | | | | | | | | | | |
| Water for | q.s | q.s | q.s | q.s | q.s | q.s | q.s | q.s | q.s | |
| Injection | | | | | | | | | | |
| % | 99.8 | 99.5 | 99.6 | 99.5 | 99.4 | 97.5 | 90.1 | 85.1 | 100.0 | 100.0 |
| Transmission | | | | | | | | | | |
| Viscosity | 20.14 | 23.28 | 24.33 | 24.85 | 28.25 | 42.38 | 57.03 | 60.95 | 1.57 | 7.59 |

The values indicate that the viscosity (in cps) of the composition increases by increasing percent PVP by weight of the total polymer or by increasing the weight by weight ratio of Hydroxypropyl methylcellulose (HPMC-E4M): Polyvinylpyrrolidone (PVP IC90) from 100:0 to 0:100. At ratio from 60:40 to 20:80 ratio (HPMC-E4M: PVP K-90), synergistic viscosity was observed. Also the compositions were clear aqueous solutions having percent transmission greater than 90%.

### Example 6

Study regarding determination of concentration of the drug retained in the rabbit's aqueous humor on topical application of the developed ophthalmic composition of Example 3 above, to the rabbit eye, and comparison of the concentration-time profile so obtained with the instillation of the marketed product NYOGEL (Novartis Pharmaceuticals Ltd).

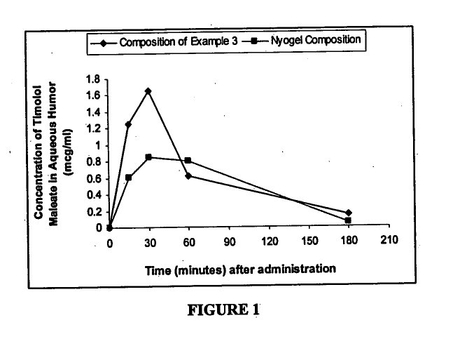

The method followed is described below and the observation is represented in FIG. 1.

Method: Two male NZ (Newzealand) rabbits weighing 1.5-3.0 Kg were taken. To both eyes of the first rabbit, a single dose of 70 μl of composition of Example 3 of the present invention, having timolol maleate in a concentration of 0.1% by weight of the composition, was instilled and to both eyes of the second rabbit, a single dose of 70 μl of the marketed product NYOGEL composition having timolol maleate in a concentration of 0.1% by weight of the composition, was instilled. At 15 min, 30 min, 1 h and 3 h post instillation of drug, animals were sacrificed by intravenous injection of thiopental sodium and aqueous humor was aspirated with insulin syringe (1 ml, BD Ultra fine) after puncturing the anterior chamber at the limbus and the samples were stored at −70° C. till analysis. These aqueous humor samples were analyzed by HPLC and the data obtained for various compositions was compared (see FIG. 1).

On analyzing the results obtained, as represented by FIG. 1, it is clear that composition of the present invention (Example 3—having timolol maleate in a concentration of 0.1% by weight of the composition) is able to provide comparatively much higher concentration of the drug; viz. a better Cmax and AUC; at the site of action, and the concentration is sustained for a much longer period of time in the desired therapeutic range, as compared to that provided by the marketed product NYOGEL (which is also a once-a-day preparation and having timolol maleate in a concentration of 0.1% by weight of the composition). Thus, the compositions of the present invention prove to be very effective ophthalmic compositions for once-a-day instillation, suitable for glaucoma therapy.

### Example 7

An ophthalmic composition for once-a-day instillation, according to the present invention is shown in Table 6.

TABLE 6

| S. | | Quantity (% w/v) | | | |
|---|---|---|---|---|---|
| No. | Ingredients | HPMC:PVP K90-20:80 | | | |
| 1. | Timolol Maleate eq. to | 0.5 | 0.5 | 0.25 | 0.25 |
| | Timolol | | | | |
| 2. | Hydroxypropyl Methyl | 0.5 | 0.5 | 0.5 | 0.5 |
| | Cellulose E4M | | | | |
| 3. | Polyvinyl Pyrrolidone K 90 | 2.0 | 2.0 | 2.0 | 2.0 |
| 4. | Sodium chloride | 0.65 | 0.65 | 0.65 | 0.65 |
| 5. | Boric acid | 0.5 | 1.0 | 0.5 | 1.0 |
| 6. | Zinc Chloride | 0.0025 | 0.0025 | 0.0025 | 0.0025 |
| 7. | Tromethamine | 0.32 | 0.45 | qs | qs |
| 8. | Water for Injection | q.s | q.s | q.s | q.s |
| 9. | pH | 6.5-7.5 | | | |
| 10 | Viscosity | 37.93 | 33.75 | 34.53 | 38.72 |
| 11 | % transmission | 99.23 | 99.44 | 99.20 | 99.09 |

Hydroxypropyl Methyl Cellulose K100M (HPMC K100M) and, PVP K 30 were dissolved separately in WFI under stirring. The two polymer phases were mixed with stirring, and autoclaved at 121° C. for 20 min followed by cooling to room temperature under stirring. Stirring should be continued until all of the cloudy, swollen or gelatinized particles are dissolved (Phase I, polymer phase). Timolol Maleate, Tromethamine, sodium chloride, boric acid and zinc chloride were dissolved sequentially in WFI at 20-25° C. with continuous stirring (Phase II, drug phase). The drug phase was mixed with the solution of phase I (polymer phase) under stirring aseptically through 2.0-0.2μ filters in series. Finally the volume was adjusted to 100% with WFI and filtered under aseptic conditions through 2.0 micron filter.

Transmittance at 650 nm was found to be approximately 99.0% and the viscosity was found to be approximately 33-40 Cps.

### Example 8

An ophthalmic composition for once-a-day instillation, according to the present invention is shown in Table 7.

TABLE 7

| Ingredients | Concentration (% w/v) |
|---|---|
| Timolol Maleate equivalent to Timolol | 0.5 |
| Hydroxypropyl Methyl Cellulose K100M | 0.5 |
| Polyvinyl Pyrrolidone K 30 | 2.0 |
| Mannitol | 4.5 |
| Benzalkonium chloride Solution | 0.02 |
| Tromethamine | qs |
| Water for Injection | qs |

Hydroxypropyl Methyl Cellulose K100M (HPMC K100M) and, PVP K 30 were dissolved separately in WFI under stirring. The two polymer phases were mixed with stirring, and autoclaved at 121° C. for 20 min followed by cooling to room temperature under stirring. Stirring should be continued until all of the cloudy, swollen or gelatinized particles are dissolved (Phase I, polymer phase). Timolol Maleate, Tromethamine, and Benzalkonium chloride were dissolved sequentially in WFI at 20-25° C. with continuous stirring (Phase II, drug phase). The drug phase was mixed with the solution of phase I (polymer phase) under stirring aseptically through 2-20 glass fiber and 0.2 μm nylon filters in series. Finally the volume was adjusted to 100% with WFI and filtered under aseptic conditions through 2-20 micron filter. The % Transmittance at 650 nm was found to be 99.671 and the viscosity was found to be 28.52 cps.

EXAMPLE 9

| Ingredients | Concentration (% w/v) |
|---|---|
| Timolol Maleate equivalent to Timolol | 0.5 |
| Hydroxypropyl Methyl Cellulose K100M | 0.5 |
| Polyvinyl Pyrrolidone K 90 | 2.0 |
| Mannitol | 4.5 |
| Benzalkonium chloride Solution | 0.02 |
| Tromethamine | q.s. |
| Water for Injection | q.s. |

The ophthalmic composition of this example 9 was prepared by following the same method as described in example 8. The % Transmittance at 650 nm was found to be 99.972 and the viscosity was found to be 65.66 Cps.

EXAMPLE 10

| Ingredients | Concentration (% w/v) |
|---|---|
| Timolol Maleate equivalent to | 0.5 |
| Timolol | |
| Hydroxy ethylcellulose (Natrosol 250 | 0.5 |
| HX) | |
| Polyvinyl Pyrrolidone K 30 | 2.0 |
| Mannitol | 4.5 |
| Benzalkonium chloride Solution | 0.02 |
| Tromethamine | 0.22 |
| Water for Injection | qs |

The ophthalmic composition of this example 9 was prepared by following the same method as described in example 8.

EXAMPLE 11

| Ingredients | Concentration (% w/v) |
|---|---|
| Timolol Maleate equivalent to Timolol | 0.5 |
| Hydroxy ethylcellulose (Natrosol 250 | 0.5 |
| HX) | |
| Polyvinyl Pyrrolidone K 90 | 2.0 |
| Mannitol | 4.5 |
| Benzalkonium chloride Solution | 0.02 |
| Tromethamine | qs |
| Water for Injection | qs |

The ophthalmic composition of this example 9 was prepared by following the same method as described in example 8. The % Transmittance at 650 nm was found to be 98.792 and the viscosity was found to be 37.15.

1. An ophthalmic composition comprising therapeutically effective amount of a beta blocker and a polymeric vehicle consisting essentially of a water soluble cellulose derivative and polyvinyl pyrrolidone, wherein the composition is a clear aqueous solution having a viscosity 20 cps to 60 cps.

2. An ophthalmic composition as claimed in claim 1 wherein the viscosity is 35 cps to 50 cps and the % transmission is 95%.

3. An ophthalmic composition as claimed in claim 1 wherein the beta-blocker is timolol maleate.

4. An ophthalmic composition as claimed in claim 1 wherein water soluble cellulose derivative is selected from the group consisting of hydroxylpropylmethyl cellulose, hydroxyethyl cellulose, hydroxypropyl cellulose, methyl cellulose and mixture thereof.

5. An ophthalmic composition as claimed in claim 3 wherein hydroxylpropylmethyl cellulose having viscosity in the range of 3500 cps to 5600 cps at 20° C. for a 2% w/v aqueous solution and 10% w/v aqueous solution of the polyvinyl pyrrolidone has a viscosity in the range of 300 cps to 700 cps at 20° C.

6. An ophthalmic composition as claimed in claim 1 wherein ratio of water soluble cellulose derivative and polyvinylpyrrolidone is in the range of 60:40 to 20:80.

7. An ophthalmic composition as claimed in claim 1 wherein the polymeric vehicle is present in a concentration of 1.4% to 5.0% by weight of the composition.

8. An ophthalmic composition as claimed in claim 1, wherein the hydroxypropyl methylcellulose is present in the amount of 0.5% by weight and polyvinylpyrrolidone is present in amount of 2.0% by weight of the composition.

9. An ophthalmic composition comprising therapeutically effective amount of a beta blocker and a polymeric vehicle consisting essentially of a water soluble cellulose derivative and polyvinyl pyrrolidone, wherein the ratio of the water soluble cellulose derivative and polyvinyl pyrrolidone ranges from 60:40 to 20:80 and the concentration of the said polymer ranges from 1.4% to 5.0% by weight of the composition.

10. An ophthalmic composition comprising therapeutically effective amount of a beta blocker and a polymeric vehicle consisting essentially of a water soluble cellulose derivative and polyvinyl pyrrolidone, wherein the ratio of the water soluble cellulose derivative and polyvinyl pyrrolidone ranges from 60:40 to 20:80 and the concentration of the said polymer ranges from 1.4% to 5.0% by weight of the composition.

11. An ophthalmic composition as claimed in claim 10 wherein the water soluble polymer is selected from the group consisting of hydroxypropyl methyl cellulose, hydroxyethyl cellulose and hydroxypropyl cellulose.

12. An ophthalmic composition as claimed in claim 10 wherein the molecular weight of the polyvinyl pyrrolidone is 1,000,000 to 1,500,000.

13. An ophthalmic composition as claimed in claim 10 wherein the polymeric vehicle consist essentially of hydroxypropyl methyl cellulose of 3500 cps to 5600 cps viscosity of 2% by weight of aqueous solution and polyvinyl pyrrolidone of 300 cps to 700 cps viscosity of 10% w/v aqueous solution.
